(19) United States
(12) Patent Application Publication
(10) Pub. No.: US 2006/0229732 A1
(43) Pub. Date: Oct. 12, 2006

(54) Femoral prosthesis component

(75) Inventor: Jean-Claude Bachelier, Bieville Beuville (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/390,764

(22) Filed: Mar. 28, 2006

(30) Foreign Application Priority Data
Mar. 30, 2005 (GB) 0506398.7

(51) Int. Cl. A61F 2/36

(52) U.S. Cl. 623/22.42; 623/22.46

(57) ABSTRACT

A femoral prosthetic component of a replacement hip joint comprises a stem for fixing in a medullary cavity. A separate proximal element is provided with a neck for receiving a modular ball head or having an integral ball head. A dove tail-shaped system is provided for securing the proximal component to the stem which includes a sliding tongue and groove joint. The tongue being provided on the stem by an undercut rail which extends in a medial-lateral direction and the groove being provided in the separate proximal element. The tongue and groove being tapered along their lengths.

### BACKGROUND OF THE INVENTION

This invention relates to a femoral prosthetic component for a replacement hip joint which comprises a stem for fixing in a medullary canal, separate proximal element provided with a neck to receive a ball head or having a ball head and connections, such as tapered connectors, for securing the proximal component to the stem which will enable a modular construction so that stems and heads of different sizes and/or shapes can be mated together.

French Patents Nos. 2,626,168 and 2,721,200 both show femoral prosthetic components which have separate stems and proximal elements which can be fastened together but both constructions show the use of a tapered plug provided on the separate proximal element and which is located in a socket in the stem. Such constructions are expensive to produce and it can be difficult to remove the plug from the stem once it has been placed in position.

U.S. Pat. No. 5,336,268 shows an adjustable hip joint endoprosthesis which has an adjustable prosthesis head which includes a link element which can slide in an undercut groove on the stem. The position of the link element can be adjusted in a medial/lateral direction and is locked in position by a threaded tension pin.

U.S. Pat. No. 5,800,560 also shows an adjustable hip joint prosthesis which is multi-dimensionally adjusted on the neck portion and which includes a dovetail shaped groove in a plate which can be secured to the stem and on which is a second plate which is movable in relation to the first and which also carries a second dovetail shaped groove which is normal to the direction of the first and which carries a tapered cone to receive a ball head. Thus the construction allows adjustment in two planes and can be firmly fastened in any position by means, for example, of screws.

Neither of the above US Patent specifications shows a construction in which the undercut grooves are tapered so that the stem and separate proximal element are accurately located in a set position by the use of tapers.

The present invention is intended to provide an improved construction which is easier for the surgeon to operate.

### SUMMARY OF THE INVENTION

According to the present invention a femoral prosthetic component of a replacement hip joint comprises a stem for fixing in a medullary cavity, a separate proximal element provided with a neck to receive a ball head or having a ball head and means for securing the proximal component to the stem which include a sliding tongue and groove joint, the tongue being provided on the stem by an undercut rail which extends in a medial-lateral direction the groove being provided in the separate proximal element, and the tongue and groove being tapered along their lengths.

Thus, with this construction it is easy for the surgeon to slide the proximal element into place, and their tapered lengths allow the parts to wedge together, and it is also relatively easy to remove the proximal element if an alternative element is required by releasing the wedging action of the tapers.

Preferably the widths and/or depth of the tongue and groove are tapered along their lengths.

In one preferred construction the opposed side walls of the tongue can be inclined to each other provide a dovetail shape.

In another convenient construction the tongue and groove have co-operating cross-sections which are substantially T-shaped.

The cross arms of the T-shapes can have downwardly projecting extensions to engage co-operating troughs in the groove.

Elements for locking the stem and proximal element together are provided, for example, by means of a set screw.

The tongue can be provided as first and second lengths with a gap between them and the groove can also have first and second lengths with a gap between them to allow the grooves to be located on the tongue at an intermediate position in the length thereof and then moved lengthwise to the final located position.

The stem and/or proximal element can be made from metal, a synthetic material or a ceramic material.

The invention also includes a kit of parts to provide a modular construction of the femoral prosthetic component as set forth above and which includes two or more alternative stems and/or two or more alternative proximal elements which are of different sizes and shapes and which have appropriate sliding tongue and groove joints which can be assembled together to form said prosthetic component.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### BRIEF DESCRIPTION OF THE DRAWINGS

The invention can be performed in various ways and some embodiments will not be described by way of example and with reference to the accompanying drawings in which:

FIG. 1 is a side elevation of a femoral prosthetic component according to the present invention;

FIG. 2 is an isometric view of the separate proximal element and part of the stem of the kind shown in FIG. 1 and ready for assembly together;

FIG. 3 is a plan view of the upper end of the stem shown in FIGS. 1 and 2;

FIG. 4 is an isometric view showing the use of means for locking the parts together;

FIG. 5 is a part cross-sectional elevation on lines V-V on FIG. 4;

FIG. 6 is an exploded view showing an alternative construction;

FIG. 7 is an isometric view of a stem utilizing a T-shaped tongue rail; and

FIG. 8 is a part cross-sectional elevation of the stem as shown in FIG. 6 secured to a separate proximal element.

### DETAILED DESCRIPTION

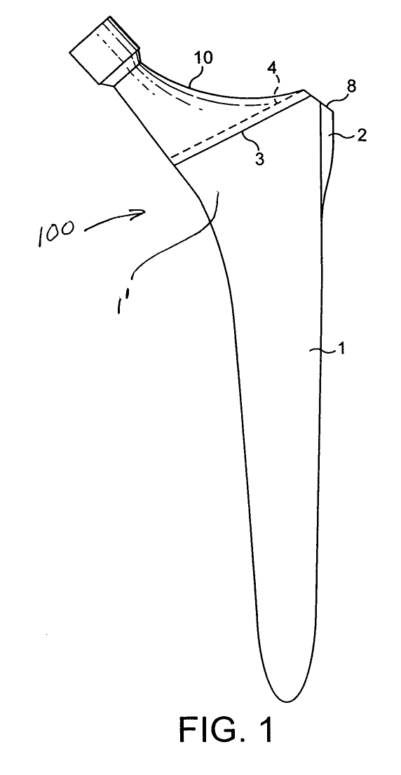

As shown in FIGS. 1 to 3 of the drawings a femoral prosthetic component of a replacement hip joint according to the present invention generally denoted as 100 comprises a distal stem 1 for fixing in a medullary cavity. The stem can be of any suitable size of shape and can be made from metal, for example a stainless steel or a composite synthetic material. In the preferred embodiment, a proximal end 1′ of the stem has a fin 2 on its lateral side to assist location.

The proximal end of the stem 1′ is shaped to provide a flat platform 3 on which is located a wedge-shaped undercut rail 4. The rail 4 tapers in width and depth from its medial to its lateral end and is undercut to that the cross-sectional width of his upper end surface 5 is greater than the cross-sectional width of its base 6 (along surface 3). The opposed side walls 7 of the rail are inclined to each other to provide a dovetail shape and rail 4 provides a tongue for a tongue and groove joint.

The upper lateral corner of the proximal stem 1′ is chamfered as indicated by reference numeral 8.

A separate proximal element 10 is provided for use with the stem 100 comprises a neck portion 11 and a tapered trunion or spigot 12 to receive a ball head (not shown) in known manner. Ball heads of this type are provided with a tapered socket to mate with spigot 12.

In an alternative construction (not shown) the ball head could be integral with proximal element 10. If a separate ball head is provided it could be made of metal, a synthetic material or ceramic material.

In the preferred embodiment the proximal element 10 has a flat planer base 13 in which is provided a tapered groove 14. The walls of the groove and the dimensions of the taper are arranged so that the groove is a sliding fit over the tongue provided by the rail 4 and the tapering sides of the walls 15 of the groove mate with side walls 7 of the rail to provide a dovetail tongue and groove joint. It will be seen that the lengthwise taper of the tongue is in a medial-lateral direction and the taper can be a Morse taper so that when the two parts are pushed into position they tend to wedge together, the inclined opposed side walls of the joint preventing any inclination to come part and again providing a taper locking connection. Typically the taper angle is between 1° and 2°.

If it is desired to take the parts apart it is merely necessary to apply a sharp blow to one of the parts in the appropriate direction to release them from the tapers.

In the construction described above the rail is tapered in both width and depth (walls 7) but, alternatively, it could only be tapered in one or the other.

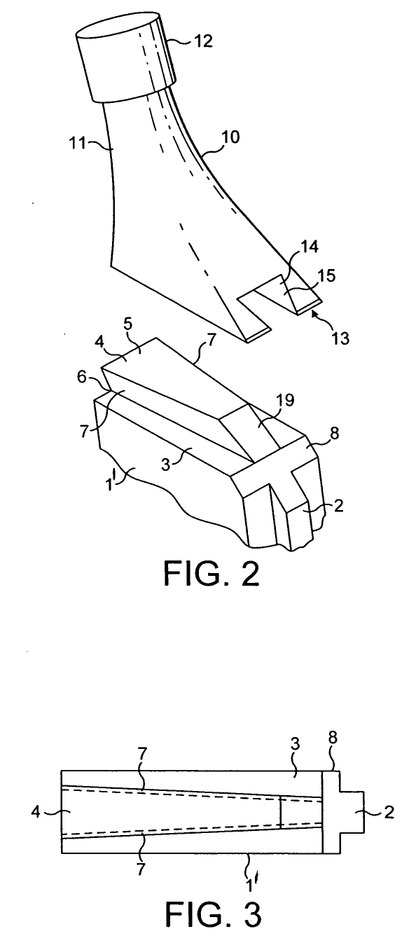

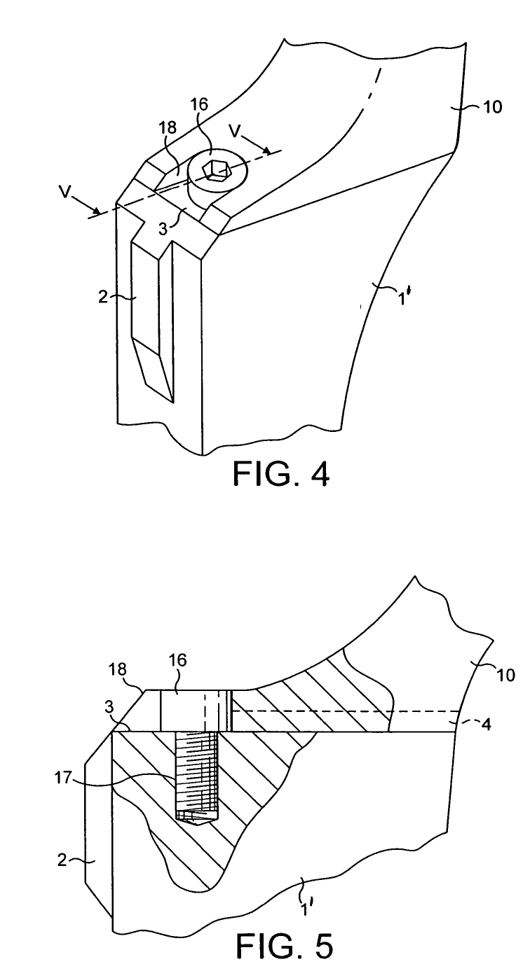

FIGS. 4 and 5 show a construction in which the same reference numerals are used to indicate similar parts to those shown in FIGS. 1 to 3. In this arrangement a locking element is included for releasably locking the separate proximal element together and to enhance the locking effect of the tapers on the tongue and groove joint. This may be achieved by use of a set screw 16 which is screwed into a threaded bore 17 in the stem. The lower part of separate proximal element 10 is cut away to provide a recess 18 in which the head of the set screw is located. The set screw 16 therefore acts as a stop to prevent proximal element 10 sliding backwards (laterally) and detaching from the stem 100. As seen in FIG. 2 the medial end of undercut rail 4 is provided with a chamfer 19. This chamfer is omitted in the construction shown in FIGS. 4 and 5 and is replaced by threaded bore 17.

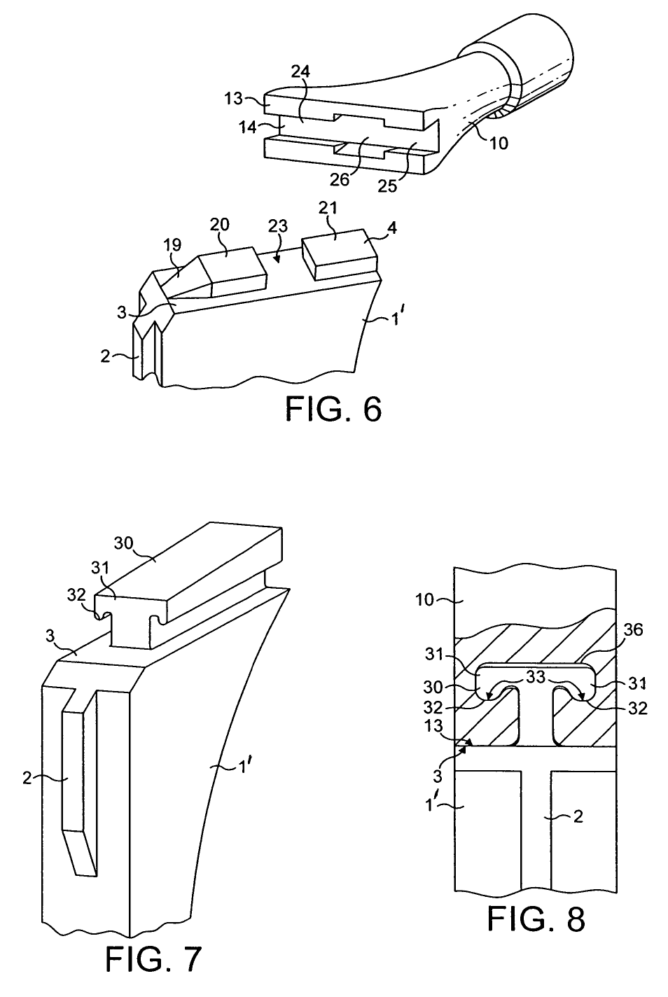

FIG. 6 shows the stem 1 and separate proximal element 10 and the same reference numbers are used to indicate similar parts as in FIGS. 1 to 3. In this arrangement however undercut rail 4, which acts as the tongue, is provided as a first medial-lateral length 20 and a second medial-lateral length 21 with a gap 23 between them. Similarly tapered groove 14 in separate proximal element 10 is also formed with a first medial-lateral length 24 and a second medial-lateral length 25 with a gap 26 between them. The dimensions of the gap 26 are arranged to be slightly larger than the length of the first length 20 of the tongue and gap 23 in the tongue is arranged to be slightly larger than the walls of the second length 25 of the groove so that proximal element 10 can be placed downwardly over stem 100 until flat planar base 13 of the proximal element engages the flat platform 3 of the stem with the proximal element located approximately halfway along rail 4. The proximal element can now be moved in a medial-lateral direction to fully engage the rail in the groove and provide the tongue and groove connection.

This construction enables the parts to be put together after the stem has been inserted in the bone and requires a short medial-lateral distance for engagement. Thus it will be appreciated that the medial-lateral dimension required is only half that of the construction shown in FIGS. 1, 2, and 3.

FIGS. 7 and 8 show another alternative construction and in which the same reference numerals are used to indicate similar parts to those shown in FIGS. 1, 2 and 3. In this construction however tapered dovetail shaped rail 4 of the construction shown in FIGS. 1, 2 and 3 is replaced by a rail 30 which is of T-shaped cross-section. The cross arms 31 of the T have downwardly projecting extensions 32 with rounded extremities. The T-shaped rail is again tapered in depth and width along its medial-lateral length.

Proximal element 10 is provided with a T-shaped co-operating groove 35 which is shaped and dimensioned to engage the tapered T-shaped rail 30 and lock in position in a similar manner to that described with regard to dovetail shaped rail 4 in the other constructions. The groove 35 is relieved at 36 to provide a small space between the top of the T of the rail and the bottom of the groove 35 and the lower corners are chamfered as will be seen from FIG. 8. With this arrangement flat planar base 13 of proximal element 10 is arranged to engage flat platform 3 on stem 100. The rounded extremities of the downwardly projecting extensions 32 of cross arms 31 engage suitably shaped rounded troughs 33 in groove 35 so that the wedging effect acting on proximal element 10 is between troughs 33 and flat planar base 13.

As mentioned above groove 35 and rail 30 are tapered in a similar manner to the construction shown in the other FIGS. 1-4 and the taper can again be a Morse taper so that when the two parts are pushed into position they tend to wedge together.

If required the positive stop provided by the set screw 16 can also be provided in the construction shown in FIGS. 7 and 8 and similarly T-shaped rail 31 and groove 35 can be provided with gaps in a similar manner to the construction shown in FIG. 6 to reduce the dimensions required to achieve engagement.

The invention provides a kit of parts which can include a number of stem elements and a number of proximal elements which could have different shapes and sizes and all of which have the sliding tongue and groove joint so that any stem can be connected to any proximal element to provide the shape required by the surgeon.

The separate proximal element can be made from any suitable material, for example metal or a synthetic plastics material.

Due to the modular arrangement proximal elements can be used which can be angled or shaped to fit the requirements of the patient and can be fitted to a suitable length of thickness of stem. This provides a very large variety of shapes and sizes.

The made up component can be used for revision surgery or the initial fitting of a prosthesis.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A modular hip prosthesis comprising:
a first stem portion having a rail extending proximally from a proximal surface thereof, said rail extending in a medial-lateral direction and tapering in cross-section from a smaller cross-section at a lateral portion to a larger cross-section at a medial portion; and
a second stem portion having a tapered groove for receiving said rail.

2. The modular hip prosthesis as set forth in claim 1 wherein said rail tapers in a thickness measured in the anterior-portion direction from a smaller thickness laterally to a larger thickness medially.

3. The modular hip prosthesis as set forth in claim 2 wherein said rail tapers in depth measured in a proximal-distal direction with said depth being greater medially than laterally along said rail.

4. The modular hip prosthesis as set forth in claim 1 wherein said rail tapers in depth measured in a proximal-distal direction with said depth being greater medially than laterally along said rail.

5. The modular hip prosthesis as set forth in claim 1 further comprising a means for locking said first stem portion to said second stem portion after said tapered rail and groove are engaged.

6. The modular hip prosthesis as set forth in claim 1 wherein said taper is at an angle between 1° and 2°.

7. The modular hip prosthesis as set forth in claim 1 wherein said rail has a chamfer extending from an outer surface of said rail to an adjacent medial-lateral surface of said first stem portion.

8. The modular hip prosthesis as set forth in claim 1 wherein said cross-section of said rail is T-shaped with a top surface of said T extending in a medial-lateral direction and being tapered in an anterior-posterior direction from a wider portion medially to a narrower portion laterally and a leg of said T establishing a depth in the proximal-distal direction increasing from a deeper portion medially to a less deep portion laterally.

9. A femoral prosthetic component of a replacement hip joint comprising a stem for fixing in a medullary cavity, a separate proximal element provided with a neck to receive a ball head or having a ball head and means for securing the proximal component to the stem which includes a sliding tongue and groove joint, the tongue being provided on the stem by an undercut rail which extends in a medial-lateral direction the groove being provided in the separate proximal element, and the tongue and groove being tapered along their lengths.

10. The femoral prosthetic component as claimed in claim 9 in which the widths and/or depths of the tongue and groove are tapered along their lengths.

11. The femoral prosthetic component as claimed in claim 9 wherein the opposed side walls of the tongue are inclined to each other to provide a dove tail shape.

12. The femoral prosthetic component as claimed in claim 9 wherein the tongue and groove have co-operating cross-sections which are substantially T-shaped.

13. The femoral prosthetic component as claimed in claim 10 wherein the cross-section of the T-shapes have downwardly projecting extensions to engage cooperating troughs in the groove.

14. The femoral prosthetic component as claimed in claim 9 including means for releasably locking the stem and proximal element together.

15. The femoral prosthetic component as claimed in claim 14 in which the locking means include a set screw.

16. The femoral prosthetic component as claimed in claim 9 in which the tongue has first and second portions with a space between said portions and the groove also has first and second portions with a space between said gap portions to allow the groove to be located on the tongue at an intermediate position along the length thereof and then moved lengthwise to the final located position.

17. The femoral prosthetic component as claimed in claim 9 in which the stem and/or proximal element are made from metal or a composite synthetic material.

18. The femoral prosthetic component as claimed in claim 9 in which a separate ball head is provided which is made of metal, a synthetic material or ceramic material.

19. A kit of components as set forth in claim 9 comprising two or more alternative stems and/or two or more alternative proximal elements which are of different sizes and/or shapes and which have appropriate sliding tongue and groove joints which can be assembled together to form said prosthetic component.
